(19) United States
(12) Patent Application Publication
(10) Pub. No.: US 2023/0105513 A1
(43) Pub. Date: Apr. 6, 2023

(54) SYSTEM AND METHOD FOR PROVIDING FERTILITY ENHANCING DIETARY RECOMMENDATIONS IN INDIVIDUALS UNDERGOING ASSISTED REPRODUCTIVE TECHNIQUES

(71) Applicant: SOCIETE DES PRODUITS NESTLE S.A., Vevey (CH)

(72) Inventors: MYRIAM AFEICHE ZEHIL, Chicago, IL (US); LOLITA BAZAROVA, Lausanne 26 (CH); ROKO PLESTINA, Morges (CH); FREDERIC RONGA, St-Cierges (CH)

(21) Appl. No.: 17/904,495

(22) PCT Filed: Feb. 16, 2021
(86) PCT No.: PCT/EP2021/053768

§ 371 Date: Aug. 18, 2022

(30) Foreign Application Priority Data
Feb. 18, 2020 (EP) 20157995.0

(51) Int. Cl. G16H 20/60

(52) U.S. Cl. CPC G16H 20/60

(57) ABSTRACT

The present invention presents new and innovative methods and systems for personalized, realtime diet and lifestyle recommendations for users that are seeking to improve their own fertility. In a preferred embodiment, the present invention relates to novel dietary recommendations for improving fertility in women undergoing Assisted Reproductive Techniques (ART), for example, these include ovulation stimulation, In Vitro Fertilization (IW), intra-uterine insemination (IUI), gamete intrafallopian transfer (GIFT), zygote intrafallopian transfer (ZIFT); or Intracytoplasmic sperm injection (ICSI).

### FIELD OF THE INVENTION

The present invention presents new and innovative methods and systems for personalized, real-time diet and lifestyle recommendations for users that are seeking to improve their own fertility.

In a preferred embodiment, the present invention relates to novel dietary recommendations for improving fertility and conception in individuals, especially women, undergoing Assisted Reproductive Techniques (ART), for example, these include ovulation stimulation, In Vitro Fertilization (IVF), intra-uterine insemination (IUI), Zygote Intrafallopian transfer (ZIFT), Gamete Intrafallopian Transfer (GIFT) and Intracytoplasmic sperm injection (ICSI).

### BACKGROUND TO THE INVENTION

Amongst individuals undergoing ART, there is not much known about what dietary components may improve pregnancy rates or intermediate ART outcomes such as embryo quality implantation success or positive pregnancy rate. Most fertility clinics suggest to stop smoking and to stop consumption of alcohol but there are usually no specific dietary recommendations other than to follow a healthy diet.

One major problem with the state of the art concerning diet and fertility is that the dietary recommendations are far too general as they do not relate to the different, specific medical conditions which affect fertility. For example, Gaskins et al. (2016), mention the importance of folic acid, soy, and dietary patterns but do not separate conception using ART from natural conception. Chiu, Chavarro, and Souter et al. (2018) differentiates by ART but does not provide doses of intake.

Another problem is that studies on fertility and diet often investigate only single nutrients at a time or food groups (Chiu et al. 2018, Chavarro et al. 2016, Showell et al. 2017) without putting them in the context of an entire diet over a day or entire meal and they do not provide recommended intake amounts to be consumed per day or for each specific, different medical condition which affects fertility.

The present invention addresses the deficiencies in the state of the art by providing new and innovative methods and systems for personalized, real-time diet and lifestyle recommendations for users.

In particular, the present invention addresses the specific condition of enhancing fertility and conception by ART, by providing a novel, consolidated dietary recommendations which combine:

specific dietary components recommended to be consumed daily
specific dietary intake amounts
specific recommendations on avoiding certain dietary components
specific recommendations on lifestyle components

### SUMMARY OF THE INVENTION

The present invention presents new and innovative methods and systems for personalized, real-time diet and lifestyle recommendations for users that are seeking to improve their own fertility.

In several embodiments, a method and system is provided that includes requesting and receiving a plurality of user attributes, comparing the plurality of user attributes to a corresponding plurality of evidence-based fertility benchmarks, determining a plurality of fertility support opportunities based on the plurality of user attributes and the comparison to the corresponding plurality of evidence-based fertility benchmarks, identifying a plurality of fertility enhancing recommendations based on the plurality of fertility support opportunities, and presenting at least one of the plurality of fertility enhancing recommendations.

In a preferred embodiment of the invention, the system and method present fertility enhancing recommendations are for increasing fertility in a individual preparing for and undergoing ART.

### DESCRIPTION OF FIGURES

FIG. 1 illustrates a system according to an embodiment of the present invention.

FIG. 2 illustrates system components according to exemplary embodiments of the present invention.

FIG. 3 illustrates system components according to an exemplary embodiment of the present invention.

FIG. 4 illustrates an example plurality of dietary recommendations for a user according to an exemplary embodiment of the present invention.

FIG. 5 illustrates a method according to an embodiment of the present invention.

FIGS. 6A and 6B illustrate a method according to an embodiment of the present invention.

### DETAILED DESCRIPTION OF THE INVENTION

To promote user fertility, it may be useful to provide a customized diet and lifestyle plan to users interested in enhancing fertility. Therefore, a customized, integrated approach is necessary to provide maximum benefit to enhance chances of conception.

One method of providing this level of personalization is to receive information from a individual regarding certain medical conditions or diseases and the current status of conception to compare with a historical evidence-based fertility database to generate recommended diet and lifestyle options that will help improve the patient's fertility based on the provided information.

In several embodiments of the invention, the patient's fertility history including the use and timing of different methods of assisted reproductive technology is documented to generate recommended diet and lifestyle options that will help improve the patient's fertility and chances of conception.

Common methods of assisted reproductive technology (ART) include:

(i) Ovulation stimulation which is the stimulation of ovulation by medication. It usually involves a medication which causes an inhibition of the negative feedback of estrogen on the pituitary gland, resulting in an increase in secretion of follicle-stimulating hormone. Medications in use for this effect are, for example, clomifene citrate, tamoxifen and letrozole. Another means of ovulation stimulation is using Follicle-stimulating hormone (FSH), directly stimulating the ovaries.
(ii) In vitro fertilization (IVF) which is fertilization outside of the body. IVF is the most effective and the most common form of assisted reproductive technology.
(iii) Intrauterine Insemination (IUI) is a fertility treatment that involves placing sperm inside a woman's uterus to facilitate fertilization. The goal of IUI is to increase the number of sperm that reach the fallopian tubes and subsequently increase the chance of fertilization.
(iv) Zygote intrafallopian transfer (ZIFT) or tubal embryo transfer. This is similar to IVF. Fertilization occurs in the laboratory and the embryo is transferred to the fallopian tube instead of the uterus.
(v) Gamete intrafallopian transfer (GIFT) involves transferring eggs and sperm into the woman's fallopian tube. Fertilization occurs in the woman's body.
(vi) Intracytoplasmic sperm injection (ICSI) is often used for couples with male factor infertility, older couples or for those with failed IVF attempts. In ICSI, a single sperm is injected into a mature egg as opposed to “conventional” fertilization where the egg and sperm are placed in a petri dish together and the sperm fertilizes an egg on its own.

An example system may be beneficial if it can provide user support throughout the entire conception journey, from the early stages of planning to the final stages of conception. As such, this example system would be useful should it provide constant, around the clock access to both virtual and personal fertility, lifestyle, nutrition, and exercise coaches. Furthermore, an example system may provide recommendations to manage anxiety, reduce stress, or provide specific supplementation, all of which are also linked to a user's fertility.

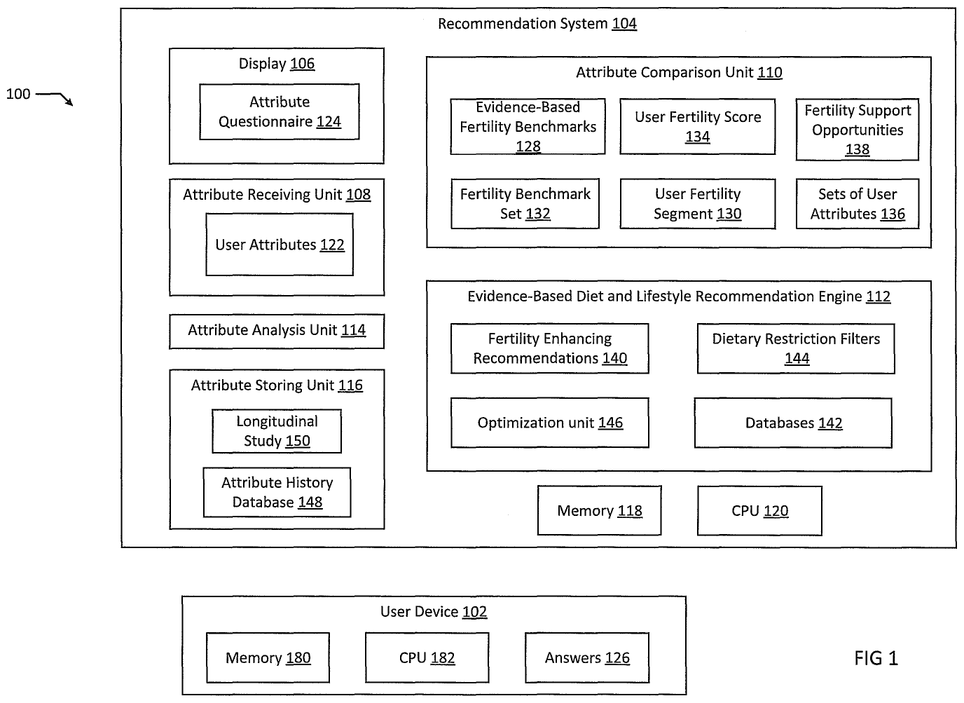

FIG. 1 illustrates a system 100 according to an embodiment of the present disclosure. The system 100 includes a user device 102 and a recommendation system 104. The user device 102 may be implemented as a computing device, such as a computer, smartphone, tablet, smartwatch, or other wearable through which an associated user can communicate with the recommendation system 104. The user device 102 may also be implemented as, e.g., a voice assistant configured to receive voice requests from a user and to process the requests either locally on a computer device proximate to the user or on a remote computing device (e.g., at a remote computing server).

The recommendation system 104 includes one or more of a display 106, an attribute receiving unit 108, an attribute comparison unit 110, an evidence-based diet and lifestyle recommendation engine 112, an attribute analysis unit 114, an attribute storing unit 116, a memory 118, and a CPU 120. Note, that in some embodiments, a display 106 may additionally or alternatively be located within the user device 102. In an example, the recommendation system 104 may be configured to receive a request for a plurality of fertility enhancing recommendations 140. For example, a user may install an application on the user device 102 that requires the user to sign up for a recommendation service. By signing up for the service, the user device 102 may send a request for the fertility enhancing recommendations 140. In a different example, the user may use the user device 102 to access a web portal using user-specific credentials. Through this web portal, the user may cause the user device 102 to request fertility enhancing recommendations from the recommendation system 104.

In another example, the recommendation system 104 may be configured to request and receive a plurality of user attributes 122. For example, the display 106 may be configured to present an attribute questionnaire 124 to the user. The attribute receiving unit 108 may be configured to receive the user attributes 122. In one example, the attribute receiving unit 108 may receive a plurality of answers 126 based on the attribute questionnaire 124, and based on the plurality of answers, determine the plurality of user attributes 122. For example, the attribute receiving unit 108 may receive answers to the attribute questionnaire 124 suggesting that the diet of the user is equivalent to the recommended dietary allowance (“RDA”) and then determine the user attributes 122 to be equivalent to the RDA, such as 500 mg/day of Vitamin C. In another example, the user device attribute receiving unit 108 may directly receive the user attributes 122 from the user device 102.

In another example, the attribute receiving unit 108 may be configured to receive the test results of a home-test kit, the results of a standardized health test administered by a medical professional, the results of a self-assessment tool used by the user, or the results of any external or third party test. Based on the results from any of these tests or tools, the attribute receiving unit 108 may be configured to determine the user attributes 122. For example, this may be measurements of the nutrient levels in blood or urine of the user which may be compared to standardized nutrient levels.

The recommendation system 104 may be further configured to compare the plurality of user attributes 122 to a corresponding plurality of evidence-based fertility benchmarks 128. For example, the attribute comparison unit 110 may be configured to determine a user fertility segment 130.

In a preferred example, the user fertility segment could be much more specific. For example, the user fertility segment could be a user preparing for or undergoing ART. The user fertility segment could be further specified as a user with a high BMI.

Furthermore, the attribute comparison unit 110 may be further configured to determine a fertility benchmark set 132 based on the user fertility segment 130. For example, if the attribute comparison unit 110 determines that a user falls into the ART user fertility segment 130, based on the plurality of user attributes 122, the attribute comparison unit 110 may select a fertility benchmark set 132 that has been created and defined according to the specific needs of a user undergoing a particular medical treatment, such as in vitro fertilization (“IVF”).

The comparison unit 110 may be further configured to select, from this determined fertility benchmark set 132, the evidence-based fertility benchmarks 128 and compare the now selected evidence-based fertility benchmarks 128 to each of the corresponding user attributes 122. For example, when the fertility benchmark set 132 has been determined, in response to the determination, the attribute comparison unit 110 may compare a user attribute 122 that represents the user's vitamin C intake to an evidence based fertility benchmark 128 that represents a benchmark vitamin C intake, determining whether the user is below, at, or above the benchmark vitamin C intake. Though this example is based on a concrete, numerical comparison, another example of a benchmark comparison may be qualitative and different depending on a person. For example, a user attribute 122 may indicate that the user is currently experiencing higher than normal levels of stress. An example benchmark related to a user stress level may indicate that an average or low level of stress is desired and thus, the user attribute 122 indicating a higher level of stress is determined to be below that of the benchmark. As different users experience differing levels of stress, even under the same circumstances, such a comparison requires a customized approach.

In addition, during the comparison from the prior example, the attribute comparison unit 110 may be configured to determine a user fertility score 134 based on the comparison between the evidence-based fertility benchmarks 128 and the user attributes 122. For example, the attribute comparison unit 110 may determine a user fertility score of 95/100 if the user attributes 122 very nearly meet all or most of the corresponding evidence-based fertility benchmarks 128. In another example, a score may be represented through lettering grades, symbols, or any other system of ranking that allows a user to interpret how well their current attributes rate amongst benchmarks. This user fertility score 134 may be presented through the display 106.

The recommendation system 104 may be further configured to determine a plurality of fertility support opportunities 138 based on the plurality of user attributes 122 and the comparison to the corresponding plurality of evidence-based fertility benchmarks 128. In one example, the attribute comparison unit 110 may determine fertility support opportunities 138 for every user attribute 122 that does not meet the corresponding evidence-based fertility benchmark. In this example, a corresponding evidence-based fertility benchmark 128 may require a user have an intake of 500 mg/day of Vitamin C, whereas the user attribute may indicate the user is only receiving 200 mg/day of Vitamin C. Therefore, the attribute comparison unit 110 may determine an increase in Vitamin C intake to be a fertility support opportunity 138.

In another example, the attribute comparison unit 110 may be configured to identify a first set of user attributes 136 comprised of each of the plurality of user attributes 122 that are below the corresponding one of the plurality of evidence-based fertility benchmarks 128 as well as identify a second set of user attributes 136 comprised of each of the plurality of user attributes 122 that are greater than or equal to the corresponding evidence-based fertility benchmarks 128. While the first set of user attributes 136 is determined similarly to the above given example, the second set of user attributes 136 differs in that, although the associated user does not appear to have a deficiency, there may be opportunities to support fertility by recommending the user maintain current practices or opportunities to further improve upon them. Accordingly, the recommendation system 104 may determine opportunities to support fertility based on which attributes 122 populate either sets 136.

The recommendation system 104 may be further configured to identify a plurality of fertility enhancing recommendations 140 based on the plurality of fertility support opportunities 138. For example, the evidence-based diet and lifestyle recommendation engine 112 may be configured to be cloud-based. The recommendation engine 112 may comprise one or more of a plurality of databases 142, a plurality of dietary restriction filters 144, and an optimization unit 146. Based on the plurality of opportunities 138, the recommendation engine 112 may identify the plurality of fertility enhancing recommendations 140 according to the one or more of plurality of databases 142, the dietary restriction filters 144, and the optimization unit 146.

In another example, the recommendation system 104 may be configured to provide continuous recommendations, based on prior user attributes. For example, the recommendation system 104 may comprise, in addition to the previously discussed elements, an attribute storing unit 116 and an attribute analysis unit 114. The attribute storing unit 116 may be configured to, responsive to the attribute receiving unit 108 receiving the plurality of user attributes 122, add the received user attributes 122 to an attribute history database 148 as a new entry based on when the plurality of user attributes 122 were received. For example, if user attributes 122 are received by the attribute receiving unit 108 on a first day, the attribute storing unit 116 will add the received user attributes 122 to a cumulative attribute history database 148 noting the date of entry, in this case the first day. Later, if user attributes 122 are received by the attribute receiving unit 108 on a second day, e.g. the next day, the attribute storing unit 116 will also add these new attributes to the attribute history database 148, noting that they were received on the second day, while also preserving the earlier attributes from the first day.

This attribute analysis unit 114 may be configured to analyze the plurality of user attributes 122 stored within the attribute history database 148, wherein analyzing the stored plurality of user attributes 122 comprises performing a longitudinal study 150. Continuing the earlier example, the attribute analysis unit 114 may perform a longitudinal study of the user attributes 122 from each of the first day, the second day, and every other collection of user attributes 122 found within the attribute history database 148. The evidence based diet and lifestyle recommendation engine 112 may be further configured to generate a plurality of fertility enhancing recommendations 140 based on at least the stored user attributes 122 found within the attribute history database 148 and the analysis performed by the attribute analysis unit 114.

In an embodiment, the attribute analysis unit 114 is further configured to repeatedly analyze the plurality of user attributes 122 stored within the attribute history database 148 responsive to the attribute storing unit 116 adding a new entry to the attribute history database 148, essentially re-analyzing all of the data within the attribute history database 148 immediately after new user attributes 122 are received. Similarly, the evidence based diet and lifestyle recommendation engine 112 may be further configured to repeatedly generate the plurality of fertility enhancing recommendations 140 responsive to the attribute analysis unit 114 completing an analysis, thereby effectively generating new fertility enhancing recommendations 140 that consider all past and present user attributes 122 each time a new set of user attributes 122 is received.

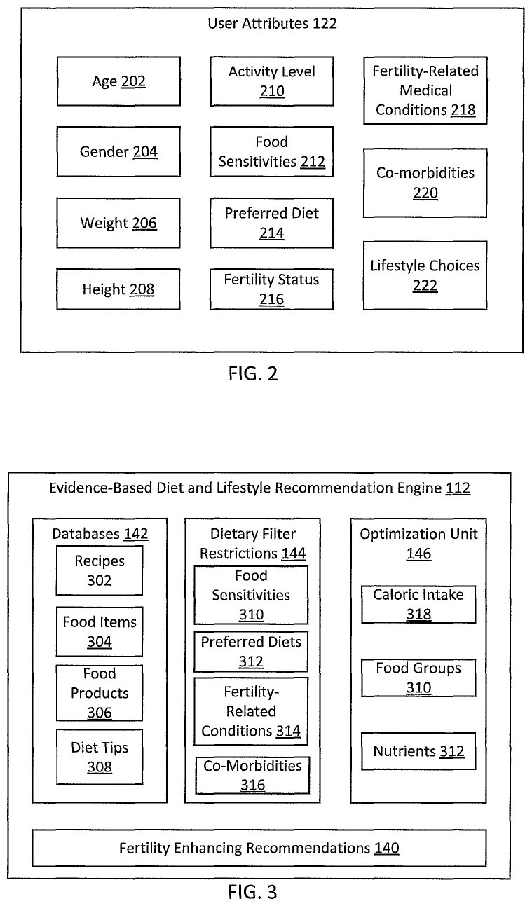

FIG. 2 illustrates an example database containing a plurality of user attributes 122. For example, the user attributes 122 may be populated by information regarding one or more of age 202, gender 204, weight 206, height 208, activity level 210, food sensitivities 212, preferred diet 214, fertility status 216, fertility-related medical conditions 218, co-morbidities 220, and lifestyle choices 222. Some examples of food sensitivities 212 include lactose, eggs, nuts, shellfish, soy, fish, and gluten sensitivities. Some non-limiting examples of a preferred diet 214 includes vegetarian, vegan, Mediterranean, kosher, halal, paleo, low carb, and low fat diets.

In a preferred embodiment, the diet is a Mediterranean diet.

Some non-limiting examples of fertility-related medical conditions 218 include polycystic ovary syndrome, premature ovarian insufficiency, endometriosis, recurring pregnancy loss, undergoing IVF, semen abnormality, misuse of anabolic steroids and protein supplements, erectile dysfunction, hormonal imbalance, low testosterone, and prostate issues.

In a preferred embodiment, the fertility-related medical condition 218 is preparing for or undergoing ART. Examples of ART include: ovulation stimulation, In Vitro Fertilization (IVF), Intrauterine Insemination (IUI), zygote intrafallopian transfer (ZIFT), gamete intrafallopian transfer (GIFT) and Intracytoplasmic sperm injection (ICSI).

Some non-limiting examples of co-morbidities 220 include diabetes, obesity, high blood pressure, high cholesterol, celiac, and heartburn. Some non-limiting examples of lifestyle choices 222 may include sleeping habits such as the typical hours of sleep per night, stress attributes such as the level of stress currently experienced by the user or typical levels of stress experienced, whether the user smokes, the number of alcoholic drinks typically consumed, exercise frequency, or any other lifestyle choices 222 that may have a bearing on fertility.

FIG. 3 illustrates an example embodiment of an evidence-based diet and lifestyle recommendation engine 112. In an example embodiment, the evidence-based diet and lifestyle recommendation engine 112 comprises a plurality of databases 142, a plurality of dietary filter restrictions 144, and an optimization unit 146. The plurality of databases 142 may include a database comprised of one or more of recipes 302, food items 304, food products 306, and diet tips 308. The dietary filter restrictions 144 may comprise filters for one or more of food sensitivities 310, preferred diets 312, fertility-related conditions 314, and co-morbidities 316. The optimization unit 146 may contain optimization rules based on one or more of caloric intake 318, food groups 310, and specific nutrients 312.

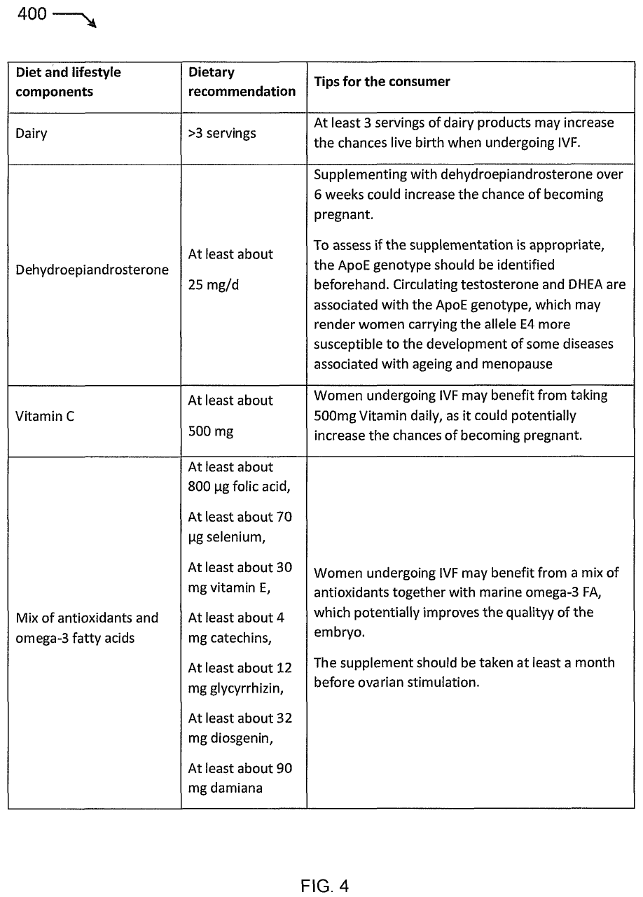

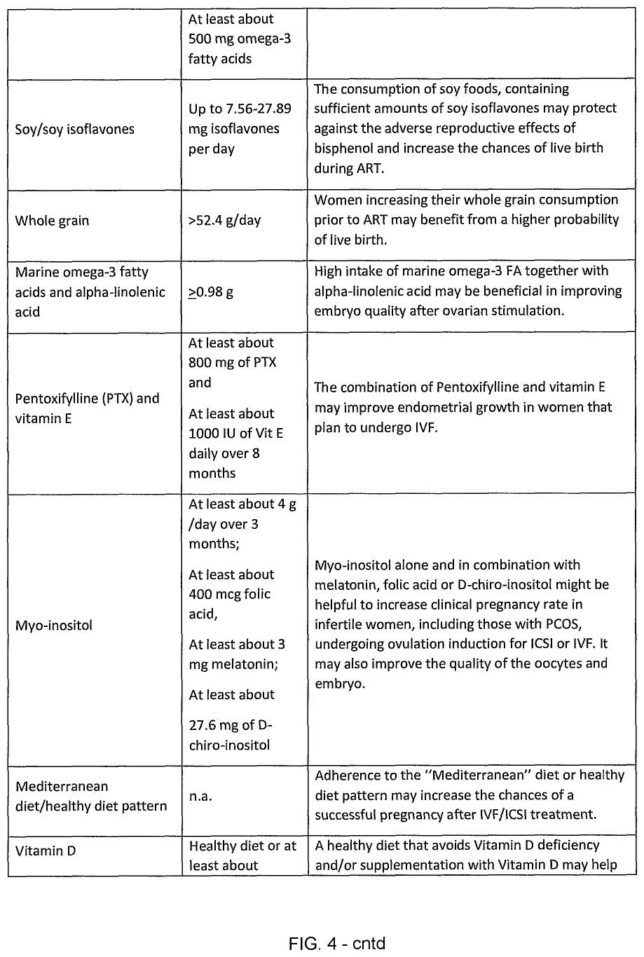

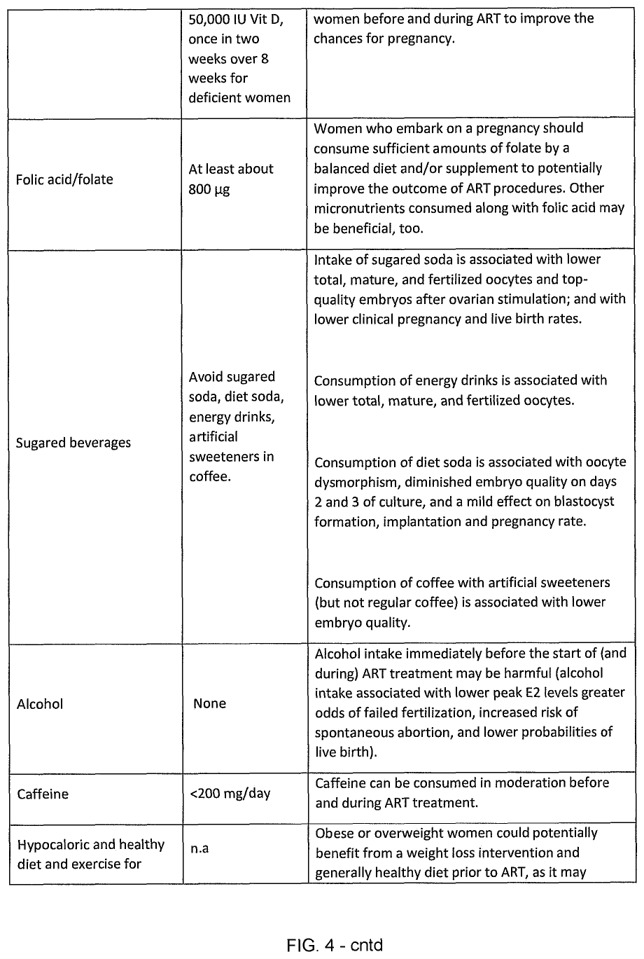

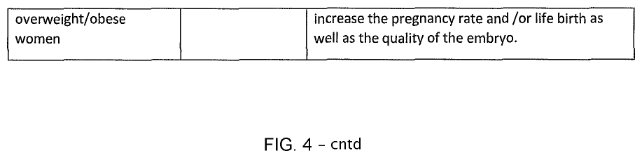

FIG. 4 illustrates an example plurality of dietary and lifestyle recommendations according to an exemplary embodiment of the present disclosure. This dietary recommendation example 400 details specific recommendations that may be presented to a user after a plurality of fertility enhancing recommendations 140 have been determined by the recommendation system 104. Specifically, example 400 details the fertility enhancing recommendations 140 as determined for a user that has a specific fertility-related medical condition 218. Specifically, example 400 represents the fertility enhancing recommendations as determined for a user that is preparing for or undergoing ART.

Other recommendations 140 may be to simply avoid, or increase, consumption of a particular food item. Similarly, the recommendations 140 may include recommendations to consume certain substances moderately or to prefer one substance over another. Though there are many different types of recommendations 140 found within the example 400, one should appreciate that any type of qualitative or quantitative recommendation may be made regarding these food items and nutrients.

Furthermore, the recommendation system 400 may generate fertility enhancing recommendations 140 that include lifestyle changes, such as altering activity level, increasing the number of hours of rest per night, taking action to reduce stress, or similar lifestyle-affecting actions. For example, a high level of stress may negatively affect a user's fertility. Such stress may derive from the relationship between the partners actively trying to conceive. Some example fertility enhancing recommendations 140 may include suggestions for methods by which the couple may decrease tensions in the relationship in order to alleviate stress. In another example, the fertility enhancing recommendations 140 may include recommendations to increase the amount of time a user rests, including sleeping habit recommendations. These recommendations may range from general recommendations, such as instruction to get more sleep, to more detailed recommendations, including specific exercise routines, specific diets and recipes, or suggested dates for visits to a medical professional.

In addition, in another embodiment, the fertility enhancing recommendations 140 generated by the recommendation system 104 may include specific recommendations for a product. For example, the recommendation system 104 may access a database containing information on a variety of supplements in the market. Then, based on its own analysis or through the use of third party research, the recommendation system 104 may analyze the different options for a specific supplement, such as Vitamin C, to determine that a specific 500 mg supplement from a first brand, Brand A, is the most beneficial supplement as compared to other 500 mg vitamin C supplements available from a second, third, and fourth brand. Such an analysis may be conducted based on the quality of the supplements, the cost of the supplements, known side effects, method of manufacture, or any other factors that may distinguish the supplement provided by one brand from a supplement provided by another brand. The recommendation system 104 may provide similar recommendations as related to food items, such as a particular type or brand of apple, and any other category of product which may require the user to select one of multiple available options.

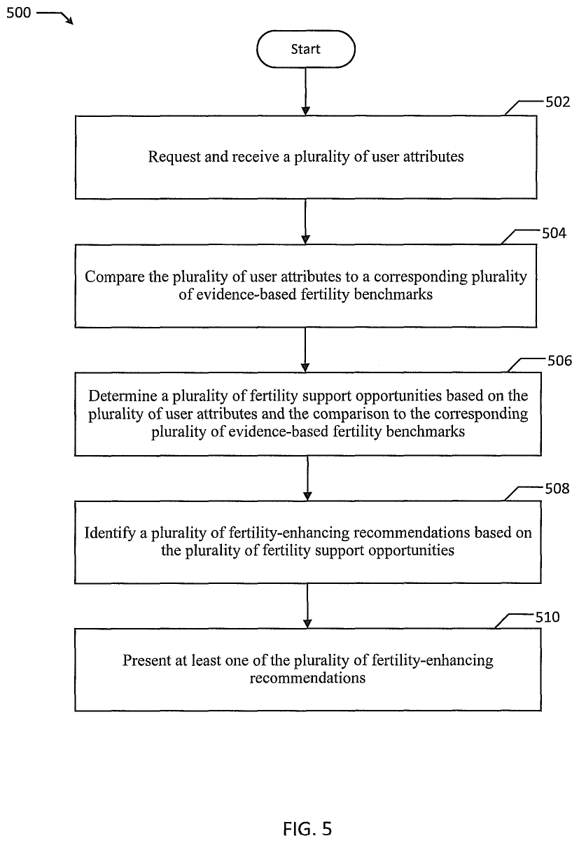

FIG. 5 illustrates an example embodiment of a method 500 of the presently disclosed method, as was discussed above in relation to the system 100. The method 500 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 500 may also be implemented by a set of instructions stored on a computer readable medium that, when executed by a processor, cause the computer system to perform the method. For example, all or part of the method 500 may be implemented by the CPU 120 and memory 118. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional.

Block 502 can include requesting and receiving a plurality of user attributes 122. For example, a display 106 may present an attribute questionnaire 124 to solicit answers 126, to which the user device 102 provides the answers 126 to then be selected as user attributes 122. In block 504, comparisons of the plurality of user attributes 122 to a corresponding plurality of evidence-based fertility benchmarks 128 may occur. Based on these comparisons, at block 506, a plurality of fertility support opportunities 138 can be determined based on the plurality of user attributes 122 and the comparison to the corresponding plurality of evidence-based fertility benchmarks 128. At block 508, an embodiment of method 500 may identify a plurality of fertility enhancing recommendations 140 based on the plurality of fertility support opportunities 138. For example, the evidence-based diet and lifestyle recommendation engine 112 may comprise a cloud-based system trained to interpret fertility support opportunities to provide recommendations 140. Lastly, at block 510, at least one of the plurality of fertility enhancing recommendations 140 can be presented.

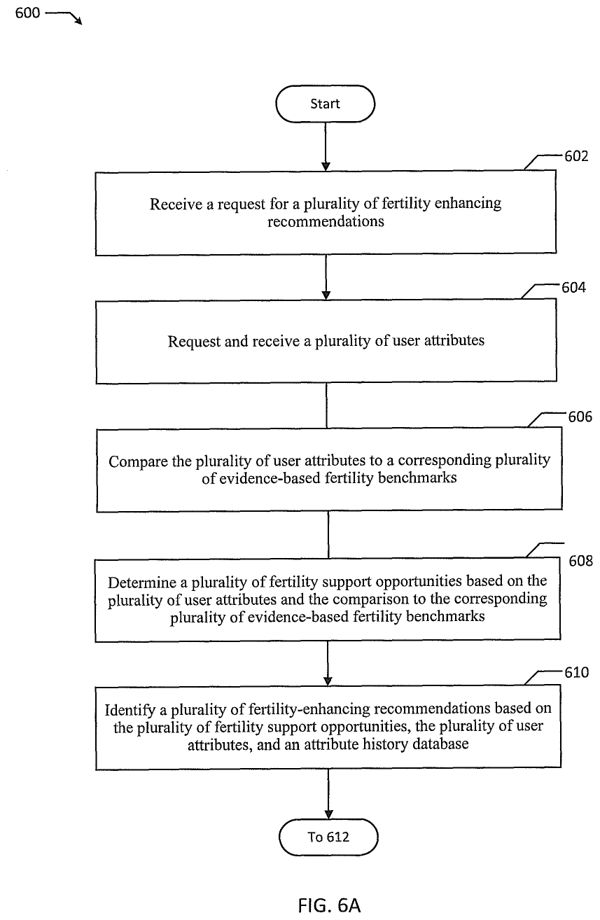

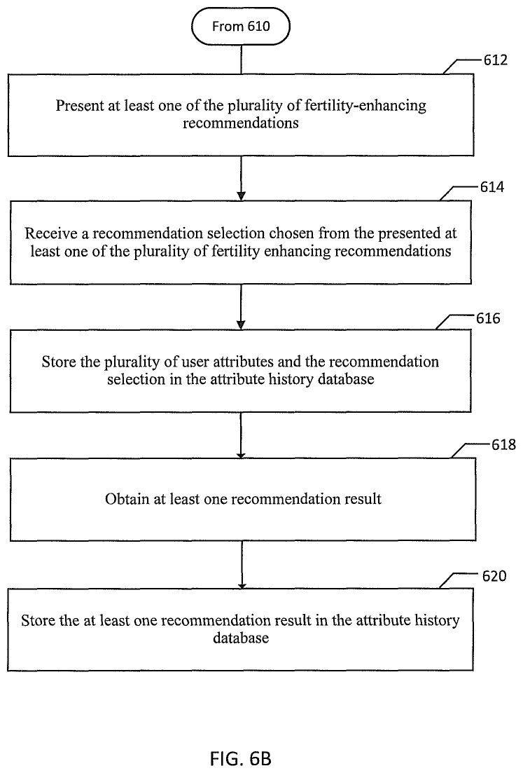

FIGS. 6A and 6B disclose an exemplary embodiment of a method 600 of the presently disclosed method. The method 600 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 600 may also be implemented by a set of instructions stored on a computer readable medium that, when executed by a processor, cause the computer system to perform the method. For example, all or part of the method 600 may be implemented by the CPU 120 and memory 118. Although the examples below are described with reference to the flowchart illustrated in FIG. 6, many other methods of performing the acts associated with FIG. 6 may be used. For example, the order of some of the blocks may be changed, certain blocks may be combined with other blocks, one or more of the blocks may be repeated, and some of the blocks described may be optional.

Block 602 may include receiving a request for a plurality of fertility enhancing recommendations 140. For example, a user may submit a request for the fertility enhancing recommendations 140 through any number of methods, including: opening an application on the user device 102, making a formal request through an application on the user device 102, submitting a request for periodic fertility enhancing recommendations 140 through the user device 102, signing into an online account through a web browser, making a formal request through a web browser, or submitting a request for periodic fertility enhancing recommendations 140 through the web browser.

At block 604, the recommendation system 104 may request and receive a plurality of user attributes 122. For example, the recommendation system 104 may present an attribute questionnaire 124 to the user. This attribute questionnaire 124 may be a standard questionnaire or a questionnaire that is customized based on known preliminary attributes, or answers to prior questions. In another example, the recommendation system 104 may request the plurality of user attributes 122 by providing a list of available home test kits, that a user may use at home. Then, after the test has been performed, the recommendation system 104 may receive the results from the test and, based on these results, determine the user attributes 122 related to such a test. For example, the home test kit may be an application to determine whether the user has become pregnant.

In another example, at block 604, the recommendation system 104 may provide a self-assessment tool. Similar to the prior example, the user will may make use of this self-assessment tool, submitting the results to the recommendation system 104. Again, based on the received results, the recommendation system 104 may determine the user attributes 122 based on the test. In yet another example, the recommendation system 104 may request the user have a standardized health test performed by a medical professional. In this example, the results of this performed health test may be submitted to the recommendation system 104, which thereby determines the user attributes 122 based on the results. Though some specific examples as to external tests have been given, these examples are non-limiting as the recommendation system 104 may be configured to receive results of any external or third party test in order to determine the corresponding user attributes 122.

In Block 606, the recommendation system 104 may be configured to compare the plurality of user attributes 122 to a corresponding plurality of evidence-based fertility benchmarks 128. For example, these evidence-based fertility benchmarks 128 may include standardized benchmarks, as in benchmarks that are given to all, regardless of individual variances. In another example, these benchmarks 128 may be customized based on a particular user's history or goals. For example, if a healthy user is trying to improve his or her fertility and the current user attributes 122 exceeds all standard evidence-based fertility benchmarks 128, the recommendation system 104 may be configured to determine a customized fertility benchmark set 132 for which the particular user should aim. In contrast, in another example, a different user that is far below a standard evidence-based fertility benchmark 128 may be compared to a different, lower benchmark value as a manner of inspiring progress and providing milestones.

The example method, at Block 608, may be configured to determine a plurality of fertility support opportunities 138 based on the plurality of user attributes 122 and the comparison to the corresponding plurality of evidence-based fertility benchmarks 128. For example, the recommendation system 104 may determine that a user attribute 122 corresponds to an above optimal stress level. Based on this comparison, the recommendation system 104 may determine a fertility support opportunity 138 to reduce stress. In another example, the recommendation system 104 may determine that the user has not yet seen a medical professional, and, as such, determine a fertility support opportunity 138 to visit a medical professional.

At Block 610, the recommendation system 104 may identify a plurality of fertility-enhancing recommendations 140 based on at least the plurality of fertility support opportunities 138. For example, the recommendation system may determine a plurality of similar prior cases by analyzing the attribute history database 148, identifying similarities between the user attributes 122 received and the plurality of prior user attributes within the attribute history database 148. For example, the recommendation system 104 may identify that the user attributes 122 detail a user with an above average BMI and other similarities that correspond to a particular group of past users and therefore the cases of those member of that particular group of past users are determined as similar prior cases.

Furthermore, in this example, the recommendation system 104 may determine a plurality of prior case results based on the plurality of similar prior cases. As detailed previously, the attribute history database 148 may comprise corresponding recommendations associated with prior user attributes, and the effectiveness of these corresponding recommendations. As such, the recommendation system 104 may analyze the corresponding recommendations and their effectiveness as associated with the particular group of past users to determine a plurality of prior case results.

Furthermore, in this example, the recommendation system 104 may determine successful recommendations and a plurality of unsuccessful recommendations based on a plurality of prior case results. For example, the recommendation system 104 may have recommended the users in that particular group of past users increase exercise levels in some cases and decrease food consumption in other cases. Based on prior case results as determined based on the attribute history database 148, the recommendation system 104 may determine that the recommendations for decreasing food consumption were not very successful, yet increasing exercise levels proved to be very successful and, as such, determines that increasing exercise levels is a successful recommendation whereas decreasing food consumption is an unsuccessful recommendation. By conducting analysis of these prior user attributes, the recommendation selection and the effectiveness of corresponding recommendations, the recommendation system 104 may identify trends associated with different subset patient populations, thereby creating and validating a plurality of lifestyle interventions. These examples of successful and unsuccessful recommendations are nonlimiting, as different groups may experience different levels of success to the same recommendations.

In addition, the recommendation system 104 may be configured to determine a plurality of fertility enhancing recommendations based on the plurality of successful recommendations and the plurality of unsuccessful recommendations. For example, the recommendation system 104 may be configured to only recommend the plurality of successful recommendations. In another example, the recommendation system 104 may still recommend any of the unsuccessful recommendations. The recommendation system 104 may make these recommendations based on any number of reasons, including a slight difference in the user attributes 122 as compared to the prior user attributes, a lack of insufficient data to support a true unsuccessful recommendation, or data supporting that, although unsuccessful, the recommendation is popular and often followed through by users. In another example, the recommendation system 104 may recommend less than all of the plurality of successful recommendations. In an example, the decision for selecting which of the plurality of recommendations to present generate may be performed by an AI.

In another example, successful recommendations may be based on guidelines associated with particular medical conditions, such as a user that is undergoing ART. In that case, these guidelines would be determined as successful recommendations.

At Block 612, the recommendation system may present at least one of the plurality of fertility-enhancing recommendations 140. At Block 614, the recommendation system 104 may receive a recommendation selection chosen from the presented at least one of the plurality of fertility enhancing recommendations 140. For example, a user may be presented with three fertility-enhancing recommendations 140, to drink less alcohol, to increase exercise, and to reduce caffeine consumption to less than 200 mg per day. The user may select one, two, or all three of these options. In an example, the user may use the user device 102 to select the fertility-enhancing recommendations 140 to increase exercise and to reduce caffeine consumption. As such, the recommendation system 104 receives, from the user device 102, these two selected recommendations as the recommendation selection. In another example, the user may not select any of the presented recommendations, at which point the recommendation system 104 may generate and present a different plurality of fertility enhancing recommendations 140.

In another example, after the user reviews the presented fertility enhancing recommendations 140, the user may submit a request to contact a fertility coach. For example, the user may be undecided as to how to implement the recommendations or may simply have a question for which the user seeks an answer. In some instances, the recommendation system 104 may determine that the question for which the user seeks an answer may be adequately answered by a virtual coach, and thereby provides access to and interaction with said virtual coach. In other instances, the recommendation system 104 may determine that the question will be best handled by a personal coach, a living individual, and thereby provides access to and interaction with said personal coach.

At Block 616, the recommendation system 104 may store the plurality of user attributes 122 and the recommendation selection in the attribute history database 148. For example, the recommendation system 104 may store all user attributes 122 received on a first day, along with the recommendation selection received on that same first day. These user attributes 122 and recommendation selection may then be accessed by the recommendation system 104 in the future when analyzing the attribute history database 148.

The recommendation system 104 may obtain at least one recommendation result at Block 618. In an example, the user may submit a recommendation result through the user device 102. This result may include a qualitative or quantitative rating as selected by the user. In another example, the recommendation system 104 may receive a future plurality of user attributes 122 and, at that time, compare the received future user attributes with the previously received user attributes, now prior user attributes, within the attribute history database 148. Based on this comparison, the recommendation system 104 may determine a recommendation result, such as decreased or increased BMI. After obtaining this recommendation result, the recommendation system 104 may store the at least one recommendation result in the attribute history database 148, corresponding to the prior recommendation selection. This recommendation system 104 may then await another request for fertility enhancing recommendations 140, and at that time, perform the method 600 again at Block 602.

Such an example method as disclosed in FIGS. 6A and 6B allow for the continuous, customized, integrated recommendation system 104 to endlessly improve upon recommendations as the attribute history database 148 grows in size. Through this growth, the recommendation system 104, and in some embodiments, the evidence-based diet and lifestyle recommendation engine 112, will have an ever-expanding set of data from which it can derive fertility enhancing recommendations 140, with increasing particularity regarding what users receive which recommendations.

In another aspect, a method of treatment may comprise using any of the above described systems or methods to generate any one or more of the fertility enhancing recommendations 140, diet and lifestyle recommendations, or specific supplementation recommendations. Furthermore, the method of treatment may comprise administering a treatment based on at least the any of one or more of the fertility enhancing recommendations 140, diet and lifestyle recommendations, or specific supplementation recommendations to a user. For example, when the recommendation system 104 determines a fertility enhancing recommendation 140 that comprises increasing a user's vitamin C intake from 200 mg/day to 500 mg/day by way of a 300 mg vitamin C supplement, an example method of treatment may comprise administering a treatment comprising a 300 mg vitamin C supplement to the user each day.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1: Dietary Recommendations for Individuals Preparing for or Undergoing ART

The following table describes the daily dietary recommendations for the individual's diet.

| | | Supplements | | |
|---|---|---|---|---|
| Diet and lifestyle | Dietary | or Diet (or | | |
| components | recommendation | both) | Tips for the consumer | Reference |
| Dairy | >3 servings | Diet | Women could potentially | [1] |
| | | | benefit from about 3 | |
| | | | servings of dairy products, | |
| | | | as it may increase the | |
| | | | chances of live birth when | |
| | | | undergoing IVF. | |
| Dehydro- | At least about | Supplement | Women with premature | [2] |
| epiandrosterone | 25 mg/d | | ovarian aging may benefit | |
| | | | from supplementing | |
| | | | dehydroepiandrosterone | |
| | | | over 6 weeks. It could | |
| | | | potentially increase the | |
| | | | chance of becoming | |
| | | | pregnant. | |
| | | | To assess if the | |
| | | | supplementation is | |
| | | | appropriate, the genotype | |
| | | | should be identified | |
| | | | beforehand. Circulating | |
| | | | testosterone and DHEA are | |
| | | | associated with the ApoE | |
| | | | genotype, which may | |
| | | | render women carrying the | |
| | | | allele E4 more susceptible | |
| | | | to the development of | |
| | | | some diseases associated | |
| | | | with ageing and | |
| | | | menopause | |
| Vitamin C | At least about | Supplement | Women undergoing IVF | [3] |
| | 500 mg | | may benefit from taking | |
| | | | 500mg Vitamin daily, as it | |
| | | | could potentially increase | |
| | | | the chances for becoming | |
| | | | pregnant. | |
| Mix of antioxidants | At least about | Supplement | Women undergoing IVF | [4] |
| and omega-3 | 800 μg folic | | may benefit from a mix of | |
| fatty acids | acid, | | antioxidants together with | |
| | At least about | | marine omega-3 FA, which | |
| | 70 μg selenium, | | potentially improves the | |
| | At least about | | quality of the embryo. | |
| | 30 mg vitamin E, | | The supplement should be | |
| | At least about | | taken at least a month | |
| | 4 mg catechins, | | before ovarian stimulation. | |
| | At least about | | | |
| | 12 mg | | | |
| | glycyrrhizin, | | | |
| | At least about | | | |
| | 32 mg diosgenin, | | | |
| | At least about | | | |
| | 90 mg damiana | | | |
| | At least about | | | |
| | 500 mg | | | |
| | omega-3 FA | | | |
| Soy/soy isoflavones | Up to 7.56- | Diet | The consumption of soy | [5, 6] |
| | 27.89 mg | | foods, containing sufficient | |
| | isoflavones | | amounts of soy isoflavones | |
| | per day | | may protect against the | |
| | | | adverse reproductive | |
| | | | effects of bisphenol and | |
| | | | increase the chances of live | |
| | | | birth during ART. | |
| Whole grain | >52.4 g/day | Diet | Women increasing their | [7, 8] |
| | | | whole grain consumption | |
| | | | prior to ART may benefit | |
| | | | from a higher probability of | |
| | | | live birth. | |
| Marine omega-3 | ≥0.98 g | Diet | High intake of marine | [9, 10] |
| fatty acids | | | omega-3 FA together with | |
| and alpha- | | | alpha-linolenic acid may be | |
| linolenic acid | | | beneficial in improving | |
| | | | embryo quality after | |
| | | | ovarian stimulation. | |
| Pentoxifylline | At least about | Supplement | The combination of | [11, 12] |
| (PTX) and | 800 mg of PTX | | Pentoxifylline and vitamin | |
| vitamin E | and at least | | E may improve | |
| | about 1000 IU | | endometrial growth in | |
| | of Vit E daily | | women that plan to | |
| | over 8 months | | undergo IVF. | |
| Myo-inositol | At least about | Supplement | Myo-inositol alone and in | [13-15] |
| | 4 g/day over 3 | | combination with | |
| | months; | | melatonin, folic acid or D- | |
| | At least about | | chiro-inositol might be | |
| | 400 mg folic | | helpful to increase clinical | |
| | acid, | | pregnancy rate in infertile | |
| | At least about | | women, including those | |
| | 3 mg melatonin; | | with PCOS, undergoing | |
| | At least about | | ovulation induction for ICSI | |
| | 27.6 mg of D- | | or IVF. It may also improve | |
| | chiro-inositol | | the quality of the oocytes | |
| | | | and embryos. | |
| Mediterranean | n.a. | Diet | Adherence to the | [16-19] |
| diet/healthy | | | “Mediterranean” diet or | |
| diet pattern | | | healthy diet pattern may | |
| | | | increase the chances of a | |
| | | | successful pregnancy after | |
| | | | IVF/ICSI treatment. | |
| Vitamin D | Healthy diet or | Both | A healthy diet that avoids | [8, 20-22] |
| | at least about | | Vitamin D deficiency | |
| | 50,000 IU Vit | | and/or supplementation | |
| | D, once in two | | with Vitamin D may help | |
| | weeks over 8 | | women before and during | |
| | weeks for | | ART to improve the | |
| | deficient women | | chances for pregnancy. | |
| Folic acid/folate | At least about | Diet and | Women who embark on a | [4, 16, 17, |
| | 800 μg | Supplement | pregnancy should consume | 23-26] |
| | | | sufficient amounts of folate | |
| | | | by a balanced diet and/or | |
| | | | supplement to potentially | |
| | | | improve the outcome of | |
| | | | ART procedures. Other | |
| | | | micronutrients consumed | |
| | | | along with folic acid may | |
| | | | be beneficial, too. | |
| | | | Intake of sugared soda is | |
| | | | associated with lower total, | |
| | | | mature, and fertilized | |
| | | | oocytes and viable | |
| | | | embryos after ovarian | |
| | | | stimulation; and with lower | |
| | | | clinical pregnancy and live | |
| | | | birth rates. | |
| Sugared beverages | Avoid sugared | Diet | Consumption of energy | [27, 28] |
| | soda, diet | | drinks is associated with | |
| | soda, energy | | lower total, mature, and | |
| | drinks, | | fertilized oocytes. | |
| | artificial | | Consumption of diet soda | |
| | sweeteners in | | is associated with oocyte | |
| | coffee. | | dysmorphism, diminished | |
| | | | embryo quality on days 2 | |
| | | | and 3 of culture, and a mild | |
| | | | effect on blastocyst | |
| | | | formation, implantation | |
| | | | and pregnancy rate. | |
| | | | Consumption of coffee | |
| | | | with artificial sweeteners | |
| | | | (but not regular coffee) is | |
| | | | associated with lower | |
| | | | Embryoquality. | |
| Alcohol | None | Diet | Alcohol intake immediately | [29, 30] |
| | | | before the start of (and | |
| | | | during) ART treatment may | |
| | | | be harmful (alcohol intake | |
| | | | associated with lower peak | |
| | | | E2 levels greater odds of | |
| | | | failed fertilization, | |
| | | | increased risk of | |
| | | | spontaneous abortion, and | |
| | | | lower probabilities of live | |
| | | | birth). | |
| Caffeine | <200 mg/day | Diet | Caffeine can be consumed | [29-32] |
| | | | in moderation before IVF | |
| | | | treatment. | |
| Hypocaloric and | n.a | Diet | Obese or overweight | [33-41] |
| healthy diet and | | | women could potentially | |
| exercise for | | | benefit from a weight loss | |
| overweight/obese | | | intervention and generally | |
| women | | | healthy diet prior to ART, | |
| | | | as it may increase the | |
| | | | pregnancy rate and /or life | |
| | | | birth as well as the quality | |
| | | | of the embryo. | |

### REFERENCES

1. Afeiche, M. C., et al., Dairy intake in relation to in vitro fertilization outcomes among women from a fertility clinic. Hum Reprod, 2016. 31(3): p. 563-71.
2. Weghofer, A., et al., The impact of androgen metabolism and FMR1 genotypes on pregnancy potential in women with dehydroepiandrosterone (DHEA) supplementation. Hum Reprod, 2012. 27(11): p. 3287-93.
3. Crha, I., et al., Ascorbic acid and infertility treatment. Cent Eur J Public Health, 2003. 11(2): p. 63-7.
4. Noun, K., et al., The Impact of a Standardized Oral Multinutrient Supplementation on Embryo Quality in in vitro Fertilization/Intracytoplasmic Sperm Injection: A Prospective Randomized Trial. Gynecol Obstet Invest, 2017. 82(1): p. 8-14.
5. Chavarro, J. E., et al., Soy Intake Modifies the Relation Between Urinary Bisphenol A Concentrations and Pregnancy Outcomes Among Women Undergoing Assisted Reproduction. J Clin Endocrinol Metab, 2016. 101(3): p. 1082-90.
6. Vanegas, J. C., et al., Soy food intake and treatment outcomes of women undergoing assisted reproductive technology. Fertil Steril, 2015. 103(3): p. 749-55 e2.
7. Gaskins, A. J., et al., Maternal whole grain intake and outcomes of in vitro fertilization. Fertil Steril, 2016. 105(6): p. 1503-1510 e4.
8. Chiu, Y. H., J. E. Chavarro, and I. Souter, Diet and female fertility: doctor, what should I eat? Fertility and Sterility, 2018. 110(4): p. 560-569.
9. Chiu, Y. H., et al., Serum omega-3 fatty acids and treatment outcomes among women undergoing assisted reproduction. Hum Reprod, 2018. 33(1): p. 156-165.
10. Hammiche, F., et al., Increased preconception omega-3 polyunsaturated fatty acid intake improves embryo morphology. Fertil Steril, 2011. 95(5): p. 1820-3.
11. Showell, M. G., et al., Antioxidants for female subfertility. Cochrane Database Syst Rev, 2017. 7: p. CD007807.
12. Acharya, S., E. Yasmin, and A. H. Balen, The use of a combination of pentoxifylline and tocopherol in women with a thin endometrium undergoing assisted conception therapies—a report of 20 cases. Hum Fertil (Camb), 2009. 12(4): p. 198-203.
13. Colazingari, S., et al., The combined therapy myo-inositol plus D-chiro-inositol, rather than D-chiro-inositol, is able to improve IVF outcomes: results from a randomized controlled trial. Arch Gynecol Obstet, 2013. 288(6): p. 1405-11.
14. Pacchiarotti, A., et al., Effect of myo-inositol and melatonin versus myo-inositol, in a randomized controlled trial, for improving in vitro fertilization of patients with polycystic ovarian syndrome. Gynecol Endocrinol, 2016. 32(1): p. 69-73.
15. Zheng, X., et al., Inositol supplement improves clinical pregnancy rate in infertile women undergoing ovulation induction for ICSI or IVF-ET. Medicine (Baltimore), 2017. 96(49): p. e8842.
16. Kermack, A. J. and N. S. Macklon, Nutritional supplementation and artificial reproductive technique (ART) outcomes. Reprod Fertil Dev, 2015. 27(4): p. 677-83.
17. Vujkovic, M., et al., The preconception Mediterranean dietary pattern in couples undergoing in vitro fertilization/intracytoplasmic sperm injection treatment increases the chance of pregnancy. Fertil Steril, 2010. 94(6): p. 2096-101.
18. Karayiannis, D., et al., Adherence to the Mediterranean diet and IVF success rate among non-obese women attempting fertility. Hum Reprod, 2018. 33(3): p. 494-502.
19. Twigt, J. M., et al., The preconception diet is associated with the chance of ongoing pregnancy in women undergoing IVF/ICSI treatment. Hum Reprod, 2012. 27(8): p. 2526-31.
20. Fatemi, F., et al., Role of vitamin E and D3 supplementation in Intra-Cytoplasmic Sperm Injection outcomes of women with polycystic ovarian syndrome: A double blinded randomized placebo-controlled trial. Clin Nutr ESPEN, 2017. 18: p. 23-30.
21. Lerchbaum, E. and T. Rabe, Vitamin D and female fertility. Curr Opin Obstet Gynecol, 2014. 26(3): p. 145-50.
22. Pacis, M. M., et al., Vitamin D and assisted reproduction: should vitamin D be routinely screened and repleted prior to ART? A systematic review. J Assist Reprod Genet, 2015. 32(3): p. 323-35.
23. Gaskins, A. J., et al., Dietary folate and reproductive success among women undergoing assisted reproduction. Obstet Gynecol, 2014. 124(4): p. 801-9.
24. Minguez-Alarcon, L., et al., Dietary folate intake and modification of the association of urinary bisphenol A concentrations with in vitro fertilization outcomes among women from a fertility clinic. Reprod Toxicol, 2016. 65: p. 104-112.
25. Servy, E. J., et al., MTHFR isoform carriers. 5-MTHF (5-methyl tetrahydrofolate) vs folic acid: a key to pregnancy outcome: a case series. J Assist Reprod Genet, 2018. 35(8): p. 1431-1435.
26. Noventa, M., et al., May Underdiagnosed Nutrition Imbalances Be Responsible fora Portion of So-Called Unexplained Infertility? From Diagnosis to Potential Treatment Options. Reprod Sci, 2016. 23(6): p. 812-22.
27. Setti, A. S., et al., Is there an association between artificial sweetener consumption and assisted reproduction outcomes? Reprod Biomed Online, 2018. 36(2): p. 145-153.
28. Machtinger, R., et al., Association between preconception maternal beverage intake and in vitro fertilization outcomes. Fertil Steril, 2017. 108(6): p. 1026-1033.
29. Abadia, L., et al., The association between pre-treatment maternal alcohol and caffeine intake and outcomes of assisted reproduction in a prospectively followed cohort. Hum Reprod, 2017. 32(9): p. 1846-1854.
30. Minguez-AlarcOn, L., J. E. Chavarro, and A. J. Gaskins, Caffeine, alcohol, smoking, and reproductive outcomes among couples undergoing assisted reproductive technology treatments. Fertility and Sterility, 2018. 110(4): p. 587-592.
31. Ricci, E., et al., Maternal and Paternal Caffeine Intake and ART Outcomes in Couples Referring to an Italian Fertility Clinic: A Prospective Cohort. Nutrients, 2018. 10(8).
32. American College of Obstetricians and Gynecologists, ACOG CommitteeOpinion No. 462: Moderate caffeine consumption during pregnancy. Obstet Gynecol, 2010. 116(2 Pt 1): p. 467-8.
33. Becker, G. F., E. P. Passos, and C. C. Moulin, Short-term effects of a hypocaloric diet with low glycemic index and low glycemic load on body adiposity, metabolic variables, ghrelin, leptin, and pregnancy rate in overweight and obese infertile women: a randomized controlled trial. Am J Clin Nutr, 2015. 102(6): p. 1365-72.
34. Braga, D. P., et al., The impact of food intake and social habits on embryo quality and the likelihood of blastocyst formation. Reprod Biomed Online, 2015. 31(1): p. 30-8.
35. Einarsson, S., et al., Weight reduction intervention for obese infertile women prior to IVF: a randomized controlled trial. Hum Reprod, 2017. 32(8): p. 1621-1630.
36. Espinos, J. J., et al., Weight decrease improves live birth rates in obese women undergoing IVF: a pilot study. Reprod Biomed Online, 2017. 35(4): p. 417-424.
37. Kasum, M., et al., The role of female obesity on in vitro fertilization outcomes. Gynecol Endocrinol, 2018. 34(3): p. 184-188.
38. Meldrum, D. R., Introduction: Obesity and reproduction. Fertil Steril, 2017. 107(4): p. 831-832.
39. Moran, L. J., et al., Altered Preconception Fatty Acid Intake Is Associated with Improved Pregnancy Rates in Overweight and Obese Women Undertaking in Vitro Fertilisation. Nutrients, 2016. 8(1).
40. Sim, K. A., et al., Weight loss improves reproductive outcomes in obese women undergoing fertility treatment: a randomized controlled trial. Clin Obes, 2014. 4(2): p. 61-8.
41. Sim, K. A., S. R. Partridge, and A. Sainsbury, Does weight loss in overweight or obese women improve fertility treatment outcomes? A systematic review. Obes Rev, 2014. 15(10): p. 839-50.

1. A method of enhancing fertility and conception for individuals preparing for or undergoing assisted reproductive technologies comprising:
requesting and receiving a plurality of user attributes;
comparing the plurality of user attributes to a corresponding plurality of evidence-based fertility benchmarks;
determining a plurality of fertility support opportunities based on at least the plurality of user attributes and the comparison to the corresponding plurality of evidence-based fertility benchmarks;
identifying a plurality of fertility enhancing recommendations based on at least the plurality of fertility support opportunities; and
presenting at least one of the plurality of fertility enhancing recommendations.

2. The method according to claim 1 wherein the individuals preparing for or undergoing assisted reproductive technologies include individuals preparing to undergo ovulation stimulation, In Vitro Fertilization (IVF), intra-uterine insemination (IUI), intra-uterine insemination, zygote intrafallopian transfer (ZIFT), gamete intrafallopian transfer (GIFT) or Intracytoplasmic sperm injection (ICSI).

3. The method according to claim 1, wherein the plurality of user attributes comprises information regarding the condition of preparing for or undergoing assisted reproductive technologies for enhancing fertility and conception and at least one of the user attributes selected from: age, gender, weight, height, activity level, food sensitivities, preferred diet, fertility status, lifestyle choices, or any co-morbidities.

4. The method according to claim 1 wherein the identifying of a plurality of fertility enhancing recommendations includes the dietary recommendations.

5. The method according to claim 1 wherein the identifying of a plurality of fertility enhancing recommendations includes recommendations for supplements.

6-7. (canceled)

8. The method according to 7 claim 1 wherein the identifying of a plurality of fertility enhancing recommendations includes dietary recommendations selected from the group of:
consuming at least 3 servings of dairy products per day;
consuming soy products or soy isoflavones each day, and
consuming whole grain products each day.

9. (canceled)

10. The method according to claim 1 wherein the identifying of a plurality of fertility enhancing recommendations includes the dietary recommendation to follow a Mediterranean diet.

11. The method according to claim 1 wherein the identifying of a plurality of fertility enhancing recommendations includes recommendations selected from the group: to avoid smoking, to avoid alcohol consumption, and/or to avoid caffeine intake greater than 200 mg/day.

12. The method according to claim 1 wherein the identifying of a plurality of fertility enhancing recommendations includes recommendations for obese individuals to follow a hypocaloric diet.

13. The method according to claim 1 wherein the identifying of a plurality of fertility enhancing recommendations based on at least the plurality of fertility support opportunities comprises:
providing the plurality of fertility support opportunities to a cloud-based artificial intelligence service; and
receiving, from the cloud-based artificial intelligence service, a plurality of fertility enhancing recommendations based on the fertility support opportunities provided to the cloud-based artificial intelligence service.

14. A computer implemented system for generating fertility enhancing recommendations for enhancing fertility and conception for individuals preparing for or undergoing assisted reproductive technologies comprising:
a memory;
a CPU;
a display configured to present an attribute questionnaire to a user;
an attribute receiving unit configured to receive a plurality of user attributes based on at least the attribute questionnaire;
an attribute comparison unit configured to compare the received plurality of user attributes to a corresponding plurality of fertility attribute benchmarks;
an evidence-based diet and lifestyle recommendation engine configured to generate a plurality of fertility enhancing recommendations based on at least the plurality of user attributes and the comparison to corresponding plurality of fertility attribute benchmarks; and
wherein the display is further configured to present at least one of the plurality of the fertility enhancing recommendations to the user.

15-21. (canceled)

22. A method of providing specific supplementation to promote fertility and conception comprising:
receiving a plurality of user dietary attributes;
comparing the plurality of user dietary attributes to a plurality of corresponding fertility dietary benchmarks;
determining a plurality of dietary deficiencies based on at least the comparison between the plurality of user dietary attributes and the plurality of corresponding fertility dietary benchmarks;
generating a plurality of specific supplementation recommendations based on the plurality of dietary deficiencies; and
presenting the plurality of supplement recommendations.

23. A method of providing specific supplementation to promote fertility and conception according to claim 22 wherein the supplement recommendation comprises
recommending administration of supplements as separate supplements or in combination comprising recommending supplements selected from the group consisting of: dehydroepiandrosterone; Vitamin C; a combination of anti-oxidants and omega-3-fatty acids; a combination of pentoxifylline and Vitamin E; myo-inositol alone or in combination with folic acid, melatonin, and D-chiro-inositol; Vitamin D; folic acid or folate.

24. A method of providing specific supplementation to promote fertility and conception according to claim 22 wherein the supplement recommendation comprises
recommending administration of supplements as separate supplements or in combination comprising recommending supplements selected from the group consisting of:
dehydroepiandrosterone in the dose of at least about 25 mg/day;
Vitamin C in the dose of at least about 500 mg/day;
Combination of anti-oxidants and omega-3-fatty acids in the dose comprising at least about 800 μg folic acid, at least about 70 μg selenium, at least about 30 mg vitamin E, at least about 4 mg catechins, at least about 12 mg glycyrrhizin, at least about 32 mg diosgenin, at least about 90 mg damiana, at least about 500 mg omega-3 fatty acids per day;
Combination of pentoxifylline and Vitamin E in the dose at least about 800 mg of pentoxifylline and at least about 1000 IU of Vitamin E daily for up to 8 months;
Myo-inositol alone in the dose of at least about 4 g/day or myo-inositol in combination with at least about 400 mg/day folic acid, at least about 3 mg/day melatonin; at least about 27.6 mg/day of D-chiro-inositol; and
Vitamin D in the dose of at least about 50,000 IU once in two weeks over 8 weeks for deficient women; or Folic acid or folate in the dose of at least about 800 μg/day.

25. The method of claim 22, wherein requesting and receiving a plurality of user attributes comprises:
providing a list of available home-test kits;
receiving a plurality of results from at least one kit from the list of available home-test kits; and
determining plurality of user attributes based on at least the plurality of results.

26. The method of claim 22, wherein requesting and receiving a plurality of user attributes comprises:
providing a self-assessment tool;
receiving a plurality of results from the self-assessment tool; and
determining the plurality of user attributes based on at least the plurality of results.

27. The method according to claim 22, wherein requesting and receiving a plurality of user attributes comprises receiving a plurality of user attributes from a standardized health test, wherein the standardized health test is administered by a medical professional.

28. The method according to claim 22 wherein the health test includes a test to determine ApoE genotype.

29-31. (canceled)
